(19) United States
(12) Patent Application Publication
(10) Pub. No.: US 2023/0218914 A1
(43) Pub. Date: Jul. 13, 2023

(54) Plasma Probe

(71) Applicant: Erbe Elektromedizin GmbH, Tuebingen (DE)

(72) Inventor: Johannes HEYM, Tuebingen (DE)

(21) Appl. No.: 18/150,327

(22) Filed: Jan. 5, 2023

(30) Foreign Application Priority Data
Jan. 7, 2022 (EP) 22150600.9

(51) Int. Cl. A61N 1/44; A61N 1/05

(52) U.S. Cl. CPC A61N 1/44; A61N 1/05; A61M 25/0082

(57) ABSTRACT

A concept for reliable and simple connection of a heat-resistant end piece (17) to a hose body (18) is described, and particularly a multiple lumen hose body (18). The connection technique is simple and reliable and leads to high quality probes having a long lifetime.

### CROSS REFERENCE TO RELATED APPLICATION

This application claims the benefit of European Patent Application No. 22150600.9, filed Jan. 7, 2022, which is incorporated herein by reference in its entirety.

TECHNICAL FIELD

The invention refers to a plasma probe, particularly for endoscopic treatment of human or animal patients as well as a method for manufacturing of such a plasma probe.

### BACKGROUND

From DE 100 30 111 B4 a generic plasma probe is known comprising a flexible hose that encloses one single lumen, the hose being provided with a mouth piece at its distal end consisting of ceramic. Inside the hose an electrical conductor is arranged that forms an electrode at its distal end located centrally in the mouth piece. During operation a suitable gas flows in distal direction through the lumen and is ionized at the electrode so that a plasma jet exits the mouth piece.

For attachment of the mouth piece to the hose, it comprises a tube shank extending into the hose.

DE 696 32 080 T2 also discloses a plasma probe having a hose body that is provided at its distal end with an end piece consisting of ceramic or PTFE. Again, the end piece comprises a shank extending into the hose in order to establish a connection between the end piece and the hose.

For centering of the electrode different measures are provided in the prior art. According to DE 696 32 080 T2, the electrode can comprise a helically wound section, the windings of which abut from the interior against the hose or the end piece and the last winding of which holds a pin-shaped electrode centrally in the end piece.

According to DE 100 30 111 B4 as an alternative a platelet-shaped element can be provided for centering the electrode, wherein the lateral edges of the platelet abut from the interior against the hose and the distal tip of which itself serves as electrode.

Both concepts reach their limits with increasing miniaturization.

Starting therefrom it is one object of the invention to provide an improved concept for configuration of the distal end of a probe.

### SUMMARY

This object is solved with a probe and a method for manufacturing a probe as described herein.

The plasma probe according to the invention comprises a hose body that encloses at least one lumen. Radially outward this lumen is limited toward the environment by means of a jacket section of the hose body. The term “radial” thereby refers to the radial direction, if the longitudinal extension of the plasma probe is considered as axial direction. The jacket section is connected with a center section by means of at least one wall section, wherein the center section can be arranged centrally or also slightly displaced from the center of the hose body. Preferably not only one wall section, but multiple wall sections are provided, e.g. three, which divide the interior of the hose body into multiple lumen and concurrently hold the center section of the hose body in the desired position, e.g. central position. The center section, the wall sections and the jacket section are preferably part of a monolithic hose body, i.e. they consist of the same material and adjoin one another without seam and joint.

At the distal end of the hose body an end piece is arranged that particularly consists of a heat-resistant material, such as a heat-resistant plastic or ceramic. The end piece comprises a central passage opening having a mouth allowing a gas or plasma exit. The mouth can be provided at the distal face side end surface of the end piece or also in a lateral area thereof. Also multiple mouths can be provided.

The end piece comprises a tube shank that extends into the hose body. For this a slit is formed between the jacket section of the hose body and the one or more wall sections, wherein the slit extends from the distal end of the hose body in proximal direction along a length into the hose body that is preferably at least as long as the length of the tube shank of the end piece, which length is to be measured in axial direction. The tube shank is inserted into this slit, whereby it displaces the wall sections radially inwardly so that they abut against the inner wall of the passage opening of the tube shank.

The wall sections of the hose body can be arranged radially or also obliquely relative to the radial direction. If they are arranged obliquely, they can easily open the slit for the tube shank in that they slightly bend. If they are arranged radially, they open the slit for the tube shank in that they bend and/or compress.

Due to the arrangement of the tube shank in the slit between the wall sections and the jacket section, the assembly of the end piece at the hose having a wall section between the jacket section and the center section is particularly simple. No material has to be removed from the interior of the hose body. It is sufficient to provide a cut in the wall section along the inner surface of the jacket section, i.e. to separate the wall section from the jacket section, e.g. by means of a cut. Due to the narrowing of the open lumen of the hose body by the tube shank of the end piece on one hand and the compressed or also inwardly bent wall sections on the other hand, a nozzle effect is created inside the tube shank of the end piece that can be advantageous for the operation as plasma probe. Particularly a too high heat introduction into the plasma probe is effectively reduced.

The hose body can be formed from a flexible, particularly bending elastic plastic, such as PTFE, PE or the like. Preferably the jacket section, the wall section (or the wall sections) and the center section are primarily shaped together thereby. For example, the hose body can be manufactured as a whole by means of extrusion.

Due to the preferred division of its interior into two, three or more lumen by means of the wall sections, a high flexibility is achieved on one hand and on the other hand it is achieved that the gas passage through the hose body is not blocked when bending the latter, also in case of small bending radii. This applies particularly, if the wall sections are arranged obliquely to the respective radial direction.

Preferably the hose body can be provided with a constant cross-section over its entire length extending from the proximal to the distal end. The distal end is the end serving for treatment facing the patient. The proximal end is the end that is to be connected to the supplying apparatus. The cross-section of the hose body is thereby referred to a surface, the surface normal of which is orientated parallel to the longitudinal direction of the hose body. The longitudinal direction extends from proximal to distal.

As already explained, the end piece can be made of a heat-resistant plastic or also of ceramic. This particularly applies for the configuration of the probe as monopolar plasma probe in which one single electrode is held in or on the center section of the hose body and serves for plasma creation. It is however also possible to make the end piece of metal and to connect it, for example, with a neutral conductor. The probe can then be realized as bipolar probe in which an electrical discharge occurs between a central electrode and the end piece. Gas flowing through the discharge can be ionized thereby so that in turn a plasma jet is created that exits the probe at the distal end.

The tube shank of the end piece serves for attachment of the end piece to the hose body. For this purpose the jacket section is placed on the outer surface of the tube shank while the wall sections abut against the wall of the passage opening at the inner side thereof. The jacket section can thereby abut against the tube shank under pretension, if the outer diameter of the tube shank is slightly larger than the inner diameter of the jacket section. In addition, an anchoring structure can be provided on the tube shank outer side, e.g. in the form of ribs, teeth or a thread.

The center section of the hose body can comprise an electrical conductor that preferably extends along the entire length of the hose body. The center section can be configured to enclose the conductor. At the proximal end of the hose body the conductor can be connected with an electrical generator by means of suitable connection means, e.g. a connector. At the distal end the electrical conductor can form an electrode or can be connected with an electrode. For example, the electrode can be configured as small tube electrode and can be supported and contacted by the conductor. It is possible to insert the tube-shaped electrode in the center section of the hose body between the conductor and the plastic material of the hose body that is thereby expanded outwardly. Under deformation of the hose, particularly its center section, the electrode is thereby held therein by means of a clamping effect. The center section can thereby be slightly expanded, whereby the flow cross-section in the tube shank is narrowed and thereby the flow velocity of the gas is increased.

Although not necessary, it is however expedient to expose the conductor prior to attachment of the electrode so that the conductor first projects in distal direction from the hose body. The tube electrode can be first moved onto this conductor and can then be moved into the center section. If a pin electrode is used, this can be omitted. The pin electrode is then simply penetrated in proximity to the conductor into the center section.

It has turned out to be advantageous, if the electrode is made of a heat-resistant electrically conductive material, such as stainless steel, hard metal, ignoble steel, tungsten, cermet or electrically conductive ceramic. The electrode can in addition be provided with a metallic coating, particularly of a metal, such as silver, the melting temperature of which is lower than the melting temperature of the electrode material. It has turned out that in so doing the electrical discharge originating from the electrode can be stabilized and the discharge footprint of which can be particularly concentrated on the distal end of the electrode.

A particularly simple and reliable method for manufacturing of the mentioned plasma probe will now be described. For this purpose first the hose body with one or multiple lumen is provided. Adjoining the inner side of the jacket section the connection between the wall section and the jacket section is separated by means of a cut. The respective cut comprises a length in axial direction of multiple millimeters and is preferably longer than the shank of the end piece measured in axial direction. Preferably this cut is carried out without material removal by means of a knife that is moved axially and/or in circumferential direction so that neither chips nor other particles (plastic particles) are created that could enter into the lumen or stick to parts of the probe and result in malfunctions later.

After production of the cut, the tube shank of the end piece is moved into the slit (the slits), wherein the wall section (or the wall sections) are displaced out of the slit. They contribute to narrowing the flow cross-section in the range of the tube section of the end piece and thus for increase of the flow velocity at this location. This supports cooling of the center section that can be used for fixation of the electrode.

### BRIEF DESCRIPTION OF THE DRAWINGS

Further details of advantageous embodiments of the invention are derived from the dependent claims and/or the figures of the drawings along with the associated description. The drawings show:

FIG. 1 an apparatus with enclosed probe in a schematic basic illustration,

FIG. 2 the distal end of the probe according to FIG. 1 in a perspective enlarged illustration,

FIG. 3 the distal end of the probe according to FIG. 2 in an exploded illustration without electrode,

FIGS. 4-6 different conditions during the provision of the hose body of the probe prior to the assembly of the end piece,

FIG. 7 the hose body in deformed condition with inserted end piece in a perspective illustration,

FIG. 8 the hose body according to FIG. 7 with assembled electrode in a perspective illustration,

FIG. 9 the distal end of the probe in a longitudinally cut illustration,

FIG. 10 the probe according to FIG. 9 with wall sections obliquely orientated relative to a radial line with view on the distal end of the probe and

FIG. 11 the probe in an illustration according to FIG. 10, however, with radially arranged compressed wall sections.

### DETAILED DESCRIPTION

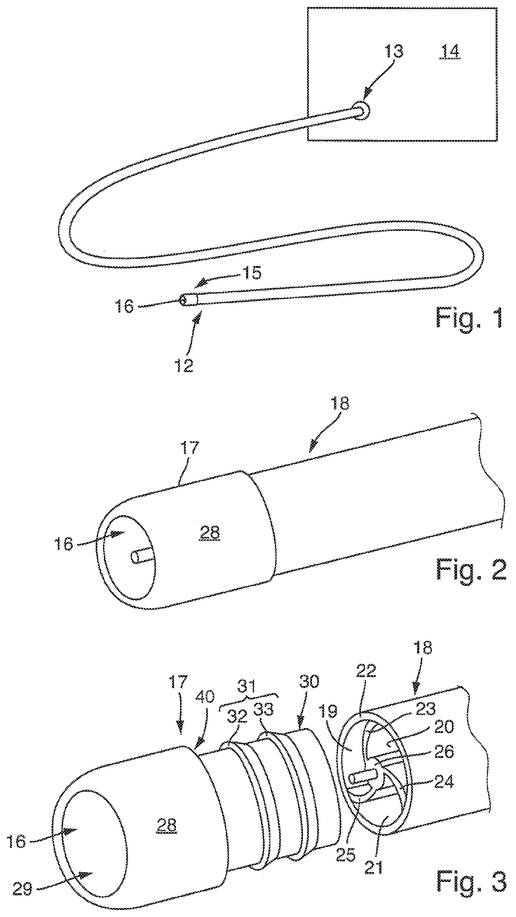

In FIG. 1 a plasma probe 12 is illustrated, the proximal end 13 of which is connected to an apparatus 14 for supply of the probe 12. The probe can be configured as plasma probe, particularly argon plasma probe. The apparatus then comprises the required components for supply of the probe 12, e.g. with radio frequency voltage and current and with a gas, particularly inert gas, particularly argon. Instead of one single apparatus 14 also multiple apparatus can be provided, each of which takes over an individual task, e.g. the supply of the probe 12 with current or the supply of the probe 12 with a suitable gas.

The probe 12 comprises a distal end 15 that lets out a plasma jet for influencing biological tissue of an animal or human patient. The probe 12 can be positioned so that the distal end 15 of probe 12 is directly located at the tissue section to be treated, e.g. through the working channel of an endoscope or through a suitable body lumen. The plasma jet of probe 12 exits from a distal opening 16 that is apparent from FIG. 2. The opening 16 is formed on an end piece 17 consisting of a heat-resistant material that is held on the hose body 18 of probe 12. While the hose body 18 consists of a flexible plastic, such as PTFE, PA12, PEBAX, polyethylene or the like, the end piece 17 is made from a typically rigid, i.e. less flexible, but in turn heat-resistant material, such as ceramic, heat-resistant plastic or the like.

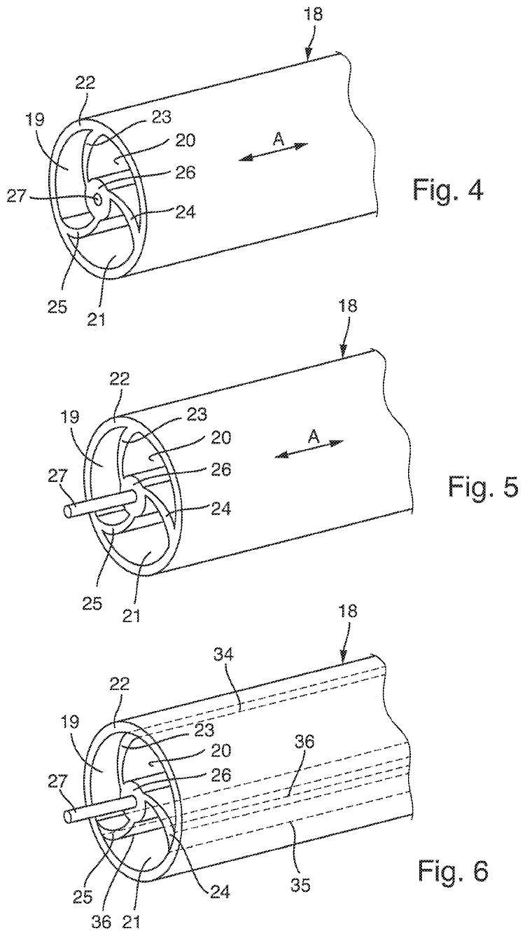

The hose body 18 encloses at least one and here three lumen 19, 20, 21, as apparent from FIG. 3, which are separated to the outward by a jacket section 22 and from one another by wall sections 23, 24, 25. The wall sections 23, 24, 25 extend from the jacket section 22 to a center section 26 with which they are connected in a seamless and monolithic manner. This configuration of the hose body 18 is particularly apparent from FIG. 4. The hose body 18 thus forms a monolithic seamless body consisting of uniform material. The center section 26 can have a substantially round or other (e.g. polygonal) cross-section and can be arranged centrally relative to the otherwise hollow cylindrical jacket section 22. Centrally in the center section 26 an electrical conductor 27 can be arranged that extends over the entire length of the hose body 18 through the center section 26. The conductor 27 can be a bare conductor or a conductor 27 insulated by means of a varnish or plastic coating. The wall sections 23, 24, 25 can be inclined relative to the radial direction, as is preferred. The wall sections 23, 24, 25 can be realized flat or curved. The radial direction extends radially away from conductor 27 that in turn extends axially. The axial direction A is indicated by an arrow in FIG. 4. The radial direction is any direction extending orthogonal thereto.

The end piece comprises a hollow body 28 on which opening 16 is configured as distal end opening, for example, as illustrated in FIGS. 2 and 3. The opening 16 of the otherwise central passage channel 29 can, however, also be arranged laterally on body 28. In addition, body 28 can comprise multiple of such openings 16 connected with the passage channel 29, e.g. distributed along its circumference. The distal end of body 28 can then be closed.

Preferably body 28 is rotationally symmetrically configured and encloses a passage channel 29 that ends at the opening 16. The passage channel 29 extends through body 28 and a tube shank 30, which adjoins body 28 and is preferably a monolithic part thereof. The tube shank 30 is preferably hollow cylindrically configured, whereby it can comprise anchoring means 31 on its outer circumference surface, such as ring beads 32, 33 having a saw-tooth-like profile, individual teeth or threads. The outer diameter of tube shank 30 is preferably at least slightly larger than the inner diameter of the cylindrical jacket section 22. On the distal end of tube shank 30 in the transition to body 28 a ring shoulder 40 is formed against which the face of jacket section 22 may abut.

For connection of end piece 17 with hose body 18 the following is carried out:

In a first step hose body 18 is provided, as illustrated in FIG. 4. The hose body 18 can be produced as continuous material by extrusion and first cut to the desired length by means of a suitable cutting tool, such as scissors or the like. At its proximal end then a supply line or a suitable connector can be connected, wherein the required space thereof plays a minor role. Special attention is paid however on the distal end and the connection of the end piece 17. For this, at least as an option, a portion is cut from the distal end of hose body 18 with a suitable cutting tool so that the conductor 27 is exposed. This simplifies the attachment of the electrode later, whereby this step can also be omitted.

The next indeed important step with relation to the attachment of the end piece 17 is the separation of the connection between the jacket section 22 and the wall sections 23, 24, 25. For this respective cuts are made along the inner circumferential surface of the jacket section 22, as indicated in FIG. 6, the cuts separating the wall sections 23, 24, 25 in the respective transition to the jacket section 22. The cuts 34-36 are indicated by dashed lines in FIG. 6. They extend over an axial distance in proximal direction into the hose body 18, whereby the distance is preferably longer than the axial length of the tube shank 30 (FIG. 3).

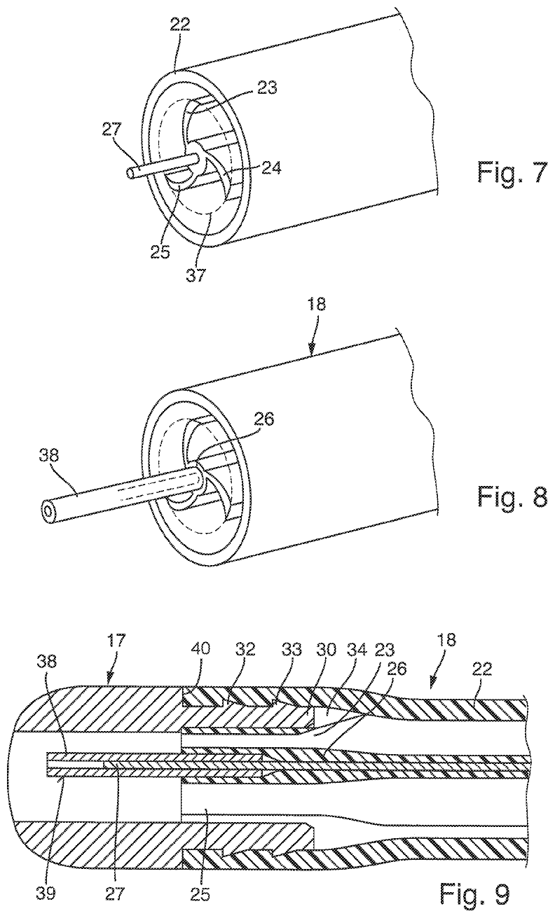

For mounting the end piece 17 on the hose body 18 the tube shank 30 is now inserted into the cuts 34-36 so that it moves between the wall sections 23, 24, 25 and the jacket section 22. Thereby the wall sections 23, 24, 25 are deformed radially inwardly, as shown in FIG. 7. The inner contour of tube shank 30 is marked by a dashed ellipse 37 in FIG. 7. The walls 23 to 25 orientated obliquely relative to the radial direction yield radially inwardly and are in so far slightly bent.

The exposed end of conductor 27 can serve itself as electrode. Additionally or alternatively, however, an electrode body 38 can be moved on the exposed end of conductor 27 and can be inserted into the center section 26 of hose body 18, as shown in FIG. 8 and particularly FIG. 9. FIG. 9 particularly illustrates the slits 34, 36 that are caused by tube shank 30 by means of deformation of the wall sections 23, 25 (as well as the cut away wall section 24 in FIG. 9). In FIG. 9 also electrode body 38 is apparent, which is configured as tube and takes the conductor 27 in the interior while it extends into the center section 26 under expansion thereof. This is independent from whether the conductor 27 has first been exposed according to FIG. 5 or whether the cuts 34, 35, 36 for mounting the end piece, i.e. for taking the tube shank 30, have been directly carried out on hose body 18 according to FIG. 4.

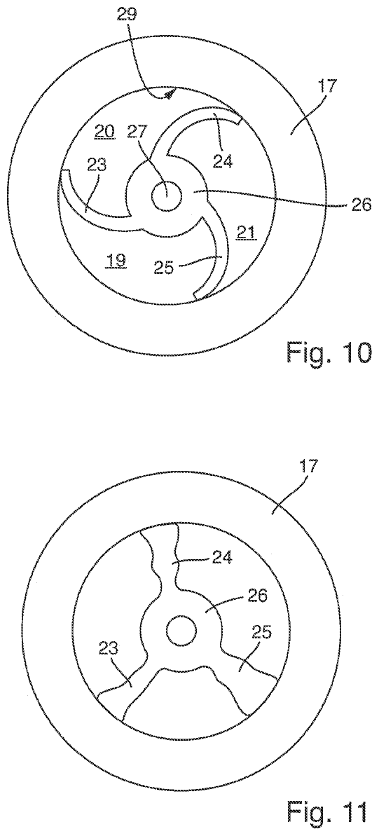

FIG. 10 illustrates the probe according to the invention again with a view into the passage channel 29. As apparent, the wall sections 23, 24, 25, which extend approximately tangentially from the center section 26 and which are cut free at the radial outer side, are displaced inwardly by the end piece 17 so that they abut against the wall of the passage channel 29. The lumen 19, 20, 21 are slightly narrowed by the tube shank and the wall sections 23, 24, 25, which slightly increases the flow velocity of the gas supplied by lumen 19, 20, 21 in the range of end piece 17.

The wall sections 23, 24, 25 do not have to be necessarily arranged obliquely relative to the radial direction and thus extend tangentially from the center section 26. They can also be arranged differently and can be orientated radially according to FIG. 11, for example. The insertion of the tube shank of end piece 17 into the slits between the wall sections 23, 24, 25 and the jacket section 22 can thereby result in a certain compression of wall sections 23 to 25, which is illustrated in FIG. 11 in a symbolic exaggerated manner. It has no influence on the details of the function of probe 12.

During operation apparatus 14 supplies lumen 19, 20, 21 of probe 12 with a suitable gas, e.g. an inert gas, such as argon. It then flows through the lumen 19, 20, 21 from the proximal end 13 of probe 12 to the distal end 15 and exits there from opening 16. By means of apparatus 14 conductor 27 and therewith electrode body 38 is applied with a high voltage relative to a neutral electrode attached to the patient. From the electrode body 38 a gas discharge originates therefore that ionizes the gas flow, so that a plasma jet exits the opening 16 via which the current supplied by generator 14 flows to the patient and via the not further illustrated neutral electrode back to generator 14. Particularly, if electrode body 38 is coated with a low-melting metal 39, particularly silver, the discharge footpoint of gas discharge concentrates on the distal end of electrode body 38. The heat introduction into the center section 26 of hose body 18 can be thereby kept low, particularly if electrode body 38 consists of a thermally low conductive material, e.g. stainless steel. The tube shank 30 narrows the open flow cross-section of lumen 19, 20, 21. The electrode body 38 widens center section 26, whereby lumen 19, 20, 21 are further narrowed. In doing so, a nozzle effect is created in the range of the tube section that contributes to a good cooling of the center section. In doing so, the plastic of center section 26 can directly contact and hold electrode body 38 without being damaged thereby.

The invention provides a concept for reliable and simple connection of a heat-resistant end piece 17 to a hose body 18, particularly a multiple lumen hose body 18. The connection technique according to the invention is simple and reliable and leads to high quality probes having a long lifetime.

### REFERENCE SIGNS

12 probe
13 proximal end of probe
14 apparatus
15 distal end
16 opening
17 end piece
18 hose body
19-21 lumen
22 jacket section
23-25 wall sections
26 center section
27 conductor
A axial direction
28 body
29 passage channel
30 tube shank
31 anchoring means
32-33 ribs (ring beads)
34-36 cuts
37 ellipse
38 electrode body
39 coating
40 ring shoulder

1. A plasma probe (12), comprising:
a hose body (18) that comprises a jacket section (22) configured in a manner limiting at least one lumen (19, 20, 21) and a center section (26), which is connected with the jacket section (22) by at least one wall section (23); and
an end piece (17) that is arranged at a distal end (15) of the hose body (18) and comprises a passage channel (29) as well as a tube shank (30) that extends into the hose body (18);
wherein between the jacket section (22) and the at least one wall section (23, 24, 25) respectively one slit (34, 35, 36) is formed that extends in a proximal direction from the distal end (15) of the hose body (18);
wherein the tube shank (30) extends into the slit (34, 35, 36).

2. The plasma probe according to claim 1, wherein the hose body (18) comprises a flexible plastic.

3. The plasma probe according to claim 1, wherein the jacket section (22), the at least one wall section (23) and the center section (26) are connected with one another in a seamless monolithic manner.

4. The plasma probe according to claim 1, wherein the hose body (18) has a constant cross-section along its entire length.

5. The plasma probe according to claim 1, wherein the end piece (17) is made of a heat-resistant plastic, a metal, or a ceramic.

6. The plasma probe according to claim 1, wherein the slit (34, 35, 36) comprises a length that is longer than the length of the tube shank (30).

7. The plasma probe according to claim 1, wherein the passage channel (29) of the tube shank (30) comprises a wall against which the at least one wall section (23) abuts with an end thereof facing the jacket section (22).

8. The plasma probe according to claim 7, cha wherein the jacket section (22) is cylindrically configured at least in an area of the tube shank (30).

9. The plasma probe according to claim 1, wherein the at least one wall section (23) is deformed inside of the tube shank (30).

10. The plasma probe according to claim 1, wherein the tube shank (30) is provided with an anchoring structure (31) on a radial outer surface of the tube shank (30).

11. The plasma probe according to claim 1, wherein an electrical conductor (27) is arranged in the center section (26).

12. The plasma probe according to claim 11, wherein an electrode body (38) is held in the center section (26).

13. The plasma probe according to claim 12, wherein the electrode body (38) is configured in a tube shape and extends between the electrical conductor (27) and the center section (26).

14. The plasma probe according to claim 12, wherein the electrode body (38) comprises a heat-resistant electrically conductive material and an electrically conductive coating (39) on a surface of the electrode body, wherein the electrically conductive coating (39) has a melting temperature which is lower than a melting temperature of the heat-resistant electrically conductive material of the electrode body (38).

15. A method for manufacturing a plasma probe,
the plasma probe comprising a hose body (18) comprising a jacket section (22) having at least one lumen (19, 20, 21) and a center section (26) that is connected with the jacket section (22) by at least one wall section (23); and
an end piece (17) configured to be arranged on a distal end (15) of the hose body (18) and that comprises a passage channel (29) as well as a tube shank (30); wherein the method comprises:
creating a slit (34) between the jacket section (22) and the at least one wall section (23) originating from the distal end (15) of the hose body (18) in a proximal direction; and
inserting the tube shank (30) in the slit (34) under displacement of the at least one wall section (23) out of the slit (34).
